(19) United States
(12) Patent Application Publication
(10) Pub. No.: US 2003/0044461 A1
(43) Pub. Date: Mar. 6, 2003

(54) Antitussive/expectorant compositions

(75) Inventors: Phuong Grace Dang, West Windsor, NJ (US); Alexander D. D'Addio, Piscataway, NJ (US)

Correspondence Address: CARELLA, BYRNE, BAIN, GILFILLAN, CECCHI,, STEWART & OLSTEIN, 6 BECKER FARM ROAD, ROSELAND, NJ, 07068, US

(73) Assignee: Carter-Wallace, Inc.

(21) Appl. No.: 09/935,322

(22) Filed: Aug. 22, 2001

(51) Int. Cl. A61K 31/7024; A61K 31/075; A61K 9/20

(52) U.S. Cl. 424/465; 514/23; 514/718

(57) ABSTRACT

Compositions consisting essentially of carbetapentane tannate and guaifenesin which are effective when administered orally for the symptomatic relief of cough associated with respiratory tract conditions such as the common cold, bronchial asthma, acute and chronic bronchitis are disclosed.

### BACKGROUND OF INVENTION

A considerable number of tannic acids occur in nature. Chemically, these acids are described as polymers of different hydroxybenzoic acids. Generally, when the term tannic acid is employed, as in the present case, the acid referred to is gallotannic acid. The internal ester of gallic acid also frequently referred to as tannin.

Tannic acid consists of an amorphous powder, glistening scales, or spongy masses varying in color from yellowish-white to light brown. Tannic acid is very soluble in water or alcohol.

Tannic acids are usually obtained from glycosides which consist of several molecules of a tannic acid in combination with glucose.

Commercially available, tannic acid, also known as tannin, has a complex non-uniform chemistry, usually contains from about 5% to about 10% water by weight, has a molecular weight of about 1700, and is typically produced from Turkish or Chinese nutgall.

Carbetapentane, known chemically as 2-[2-(diethylamino)ethoxy]ethyl 1-phenylcyclopentanecarboxylate is an antitussive compound that is described in U.S. Pat. No. 2,842,585 and is structurally related to caramiphen. Carbetapentane citrate has a melting point of 93° C. and occurs as a white powder freely soluble in water and slightly soluble in alcohol.

Carbetapentane has an atropine-like action that depresses the cough reflex by selective central nervous system depression.

Antitussive compounds in the form of their free bases as well as their salts, e.g. hydrochloride, citrate, maleate, tannate, etc., are well known. Antitussives in the form of their tannate salts are very desirable because such salts are generally stable.

Antitussives in the form of their tannate salts are typically prepared by reacting the free base, e.g. carbetapentane, etc. with tannic acid in the presence of a volatile solvent, usually isopropanol. Typically, in the conventional isopropanol route, the antitussive free base and the tannic acid will be present in the isopropanol at a concentration of about 20% based on the weight of the reaction mixture. The reaction mixture is stirred for about one hour while maintaining the mixture at 60-70° C. The reaction mixture is cooled to room temperature and then filtered, washed with isopropanol and then vacuum dried. Alternative routes to the tannate salts are described in U.S. Pat. No. 5,599,846 and 5,663,415.

Guaifenesin, known chemically as 3-(2-methoxyphenoxy)-1,2-propanediol, is a crystalline powder soluble in water and alcohol. It is indicated in the USP Drug Information as an expectorant for the symptomatic relief of cough due to colds and minor upper respiratory infections.

### THE INVENTION

It has now been found that the novel combination of carbetapentane tannate and guaifenesin produces a composition having antitussive and expectorant properties superior to the use of either compound alone. Guaifenesin has an expectorant action which increases the output of respiratory tract fluid by reducing adhesiveness and surface tension. The increased flow of less viscous secretions promotes ciliary action and facilitates the removal of mucus. This changes a dry, unproductive cough to one that is more productive and less frequent.

The compositions described herein are designed to be taken twice a day with the immediate expectorant action of guaifenesin and the prolonged antitussive action of carbetapentane tannate. The compositions of the present invention may be prepared for oral administration in the form of powders, capsules, elixirs, syrups and the preferred forms of tablets and suspensions.

Tablets containing the unique carbetapentane tannate and guaifenesin compositions of the present invention are prepared in a conventional manner by the addition of suitable pharmaceutical carriers including fillers, diluents, colorants, lubricants and the like as well as conventional and well known binding and disintegrating agents. Each tablet would contain approximately 50 to 75 mg of carbetapentane tannate and 100 to 300 mg of guaifenesin. A typical tablet composition of the present invention containing starch, dibasic calcium phosphate, colorants, magnesium stearate, methylcellulose, polygalacturonic acid, povidone and talc, as described in Example 1 which follows, is prepared by well known conventional tabletting techniques such as those disclosed in U.S. Pat. Nos. 3,018,221; 2,798,024 and 2,757,124.

### EXAMPLE 1

### Carbetapentane Tannate and Guaifenesin Tablets

| Ingredient | Milligrams per Tablet |
|---|---|
| Carbetapentane Tannate | 60.00 |
| Guaifenesin | 200.00 |
| Starch, NF | 65.00 |
| Methylcellulose, USP | 150.00 |
| Polygalacturonic Acid | 32.00 |
| Dibasic Calcium Phosphate, USP, Dihydrate | 65.00 |
| Povidone, USP | 25.00 |
| Talc, USP | 5.40 |
| FD & C Red #40 Aluminum Lake-40% | 0.35 |
| Magnesium Stearate, NF | 4.00 |
| Purified Water, USP (Deionized) | 105.00 |
| Alcohol Specially Denatured 23A 190 Proof | 35.00¹ |

¹Not present in the finished tablet product

Suspensions of the compositions of the present invention are prepared in a conventional manner such that each 5 mL (one teaspoon) contains 20 to 40 mg carbetapentane tannate and 50 to 150 mg guaifenesin. Additionally, the suspension formulations may contain colorants, natural and artificial flavors, glycerin, kaolin, magnesium aluminum silicate, methylparaben, benzoic acid, sorbic acid, pectin, purified water, saccharin, sodium hydroxide and sucrose or sorbitol. Example 2, which follows, is illustrative of a typical suspension formulation of the present invention prepared by conventional well known compounding techniques.

### EXAMPLE 2

### Carbetapetane Tannate and Guaifenesin Suspension

| Ingredient | Milligrams per 5 mL |
|---|---|
| Carbetapentane Tannate | 30.00 |
| Guaifenesin | 100.00 |
| Pectin, USP (Medium Viscosity) | 50.00 |
| Kaolin, USP (Colloidal Powder) | 1000.00 |
| Magnesium Aluminum Silicate, NF | 35.00 |
| Benzoic Acid, USP | 10.00 |
| Methylparaben, NF | 5.00 |
| Sucrose, NF | 1000.00 |
| Saccharin Sodium, USP | 2.00 |
| Glycerin, USP | 225.00 |
| Sorbic Acid | 6.00 |
| Flavor Black Currant Imitation | 0.91 |
| Flavor Strawberry with Other Natural Flavors | 2.28 |
| FD & C Red #3 Dye | 1.60 |
| Sodium Hydroxide Solution-50% | 0.30¹ |
| Purified Water, USP (Deionized) adjust to | 5 mL |

¹The quantity of Sodium Hydroxide Solution may be varied depending on the pH of the Kaolin used in the batch. Tannic acid may also be used in lieu of sodium hydroxide solution for pH adjustment. Sodium citrate, USP, Dihydrate and Citric Acid, USP, Anhydrous may also be included in the formula for pH adjustment.

For the purpose of this disclosure, a warm-blooded animal is a member of the animal kingdom possessed of a homeostatic mechanism and includes mammals and birds.

The dosage administered will be dependent on the age, health and weight of the recipient, kinds of concurrent treatment, if any, frequency of treatment and effect desired.

It should be understood that the above examples are illustrative of the best mode only of the invention herein disclosed. Given the present disclosure, it is anticipated that numerous variations will occur to those skilled in the art. A latitude of modification, substitution and change is intended and in some instances, some features of the invention will be employed without a corresponding use of other features. Accordingly, it is intended that the spirit and scope of the invention disclosed herein should be limited only by the following claims.

What is claimed is:

1. Therapeutic compositions for the symptomatic relief of cough associated with respiratory tract conditions such as the common cold, bronchial asthma, acute and chronic bronchitis in warm-blooded animals in need of such treatment said compositions comprising of pharmaceutically effective amounts of carbetapentane tannate and guaifenesin.

2. A therapeutic composition as claimed in claim 1 in tablet form.

3. A therapeutic composition as claimed in claim 1 in suspension form.

4. A method for symptomatically treating and relieving the distress of cough associated with respiratory tract conditions resulting from the common cold, bronchial asthma, acute and chronic bronchitis in warm-blooded animals which comprises orally administering to warm-blooded animals in need of such treatment a therapeutic amount of compositions consisting essentially of carbetapentane tannate and guaifenesin.

5. A method as claimed in claim 4 wherein said composition is in tablet form.

6. A method as claimed in claim 4 wherein said composition is a suspension.
